(19) United States
(12) Patent Application Publication
(10) Pub. No.: US 2009/0281614 A1
(43) Pub. Date: Nov. 12, 2009

(54) Sinus patches for replacing defective sinuses at the aortic root

(75) Inventors: Helmut Goldmann, Tuttlingen (DE); Christof Merckle, Mannheim (DE); Lisa Lippoth, Denkingen (DE)

Correspondence Address: IP GROUP OF DLA PIPER LLP (US), ONE LIBERTY PLACE, 1650 MARKET ST, SUITE 4900, PHILADELPHIA, PA, 19103, US

(73) Assignee: AESCULAP AG, a corporation of Germany, Tuttlingen/Donau (DE)

(21) Appl. No.: 12/145,539

(22) Filed: Jun. 25, 2008

(30) Foreign Application Priority Data
Jun. 27, 2007 (DE) DE102007031146.1

(51) Int. Cl. A61F 2/82

(52) U.S. Cl. 623/1.15

(57) ABSTRACT

Sinus patches for replacing defective sinuses at the aortic sinus include a flat, flexible, domed piece of wall material which, except for possibly having pleats, has a three-dimensional dome shape, which is determined by the wall material itself.

### RELATED APPLICATION

This application claims priority of German Patent Application No. 102207031146.1, filed Jun. 27, 2007, herein incorporated by reference.

### TECHNICAL FIELD

This disclosure relates to sinus patches for replacing defective sinuses at the aortic root, consisting of a flat, flexible, domed piece of wall material.

### BACKGROUND

Various techniques can be used in cardiac procedures when operating in the area around the aortic valve and the aortic root, depending on the size of the defect in this area. If the aortic valve as such is still intact, but the aortic root is damaged, aortic prostheses can be used, which already have sinuses or bulbi in the region of the aortic root. It is also possible to implant entire aortic arch prostheses with the sinuses.

An aortic root prosthesis is disclosed in WO 01/52776 A1, which has three sinuses at the end near the heart. These sinuses are cut into the required shape from a flat vascular prosthesis material. The sinus patches are then stitched with a thread along their outer edge and gathered to produce a three-dimensional dome. They are then stitched to the bottom edge of the aortic sinus root prosthesis.

### SUMMARY

We provide a sinus patch for replacing defective sinuses of an aortic root including a flat, flexible, domed piece of wall material optionally having pleats and having a three-dimensional dome shape, which is determined by the wall material itself.

### BRIEF DESCRIPTION OF THE DRAWINGS

Further characteristics of our disclosure are illustrated in the following description of preferred structures. In this case, the various characteristics can either be realized individually in their own right, or else in combination with each other.

The drawings show:

FIG. 1: a structure in the form of sections cut from aortic arch prostheses;

FIG. 2: a cross-section through lines II-II according to FIG. 1;

FIG. 3: a further structure in the form of sections cut from a toroidal-shaped vascular prosthesis;

FIG. 4: a view of another of our structures; and

FIG. 5: a view of a further structure.

### DETAILED DESCRIPTION

We provide a means to improve the application possibilities of sinus patches. We provide sinus patches being formed from a flat, flexible, domed piece of wall material which, apart from possibly having pleats, has a three-dimensional dome shape, which is determined by the wall material itself. The dome shape is an inherent characteristic of the wall. This means that the sinus patches are formed from a wall material having a three-dimensional dome shape right from the start. On the one hand, this means that the stitching and gathering stages can be dispensed with. On the other hand, it enables the shape and size of the sinus patches to be adjusted and corrected just before implantation, without having any adverse effect on the dome shape. For example, the sinus patches can be cut along the edge, a process that is not possible with the sinus patches produced by stitching and gathering. The sinus patches are particularly suitable for directly replacing defective sinuses or bulbi, i.e., for those operations where the natural aortic sinus root is still intact and only the bulbi are replaced.

In one structure, the sinus patches are sections cut from a three-dimensionally domed wall, especially a vascular prosthesis wall. The dome is preferably pre-formed. This is achieved by molding a wall material that is originally flat or pre-formed only two-dimensionally to produce a three-dimensional dome. It is also possible to pre-form the three-dimensional dome during actual production of the wall material.

In one structure, the sinus patches are therefore sections cut from a wall at the outer side of an aortic arch prosthesis having a curvature that is inherent to the material, and particularly a fixed curvature. Such cut sections already have a three-dimensional dome shape because of the shape of the aortic arch prostheses at their outer side. Another structure also provides for the sinus patches being cut from a toroidal-shaped wall, especially a vascular prosthesis wall. Here too, the three-dimensional dome shape is an inherent characteristic of the material itself.

The sinus patches may be made from a textile wall material. This may be woven or warp-knitted, for example. A wall material similar to that used for vascular prostheses is normally used. It is also possible to produce the sinus patches from a nonwoven material. Spray-bonded webs are particularly preferred in this case, especially those made from non-crosslinked or linear polyurethane. Walls made from a nonwoven material can be produced very easily so that they already have the required dome shape.

The sinus patches may be pleated in a known manner. The pleats may run in the transverse direction, such as is normally the case with vascular prostheses. It is also possible, and preferable, for the pleats to run in the longitudinal direction of the sinus patches, which normally corresponds basically to the longitudinal direction of the aortic root. Such pleats can be used to increase the dimensional stability and/or extensibility of the sinus patches.

The sinus patches are preferably longer than they are wide, such as is the case with natural sinuses. The shape is usually in the form of a coat of arms or shield. As has already been said, the exact shape can be obtained by cutting or trimming to the shape required.

The toroidal-shaped wall, especially the toroidal-shaped, vascular prosthesis segment, may be made from a textile or nonwoven material. It is preferably in the form of a woven textile substrate. In this case, a toroidal-shaped, woven tube, when looked at over the circumference of the tube, can have a constant number of warp threads in the region of the dome. This means that the dome is formed by the distances between the warp threads becoming larger. However, with a toroidal-shaped tube, it is also possible to produce the dome by feeding in additional warp threads in the region of the dome, i.e., the toroidal-shaped tubular segment exhibits an increasing and then a decreasing number of warp threads over the circumference.

In another structure, it is possible to start with a cylindrical or conical tube, which is then converted into the desired dome shape by shrinking and/or stretching the wall material, especially a textile wall material, whereby the dome shape is stabilized by a fixing process, for example by heat-setting.

As had already been said, the dome can also be produced by forming the sinus patches by depositing material onto a three-dimensionally shaped core. This type of forming process is especially suitable for sinus patches formed as spray-bonded webs.

The wall material of the sinus patches is preferably porous. As with vascular prostheses, this porosity ensures that the connective tissue can grow in. Furthermore, the sinus patches may also be coated or impregnated, especially with a resorbable material, to seal the wall material.

The sinus patches may be present in various sizes, depending on the size of the sinuses that have to be replaced, for example. The sinus patches may also have different widths for basically the same length. This is an advantage if a patient has an asymmetrical aortic valve, in which the individual sinuses are of a different size. Here too, it is possible to make the necessary adjustment before implantation.

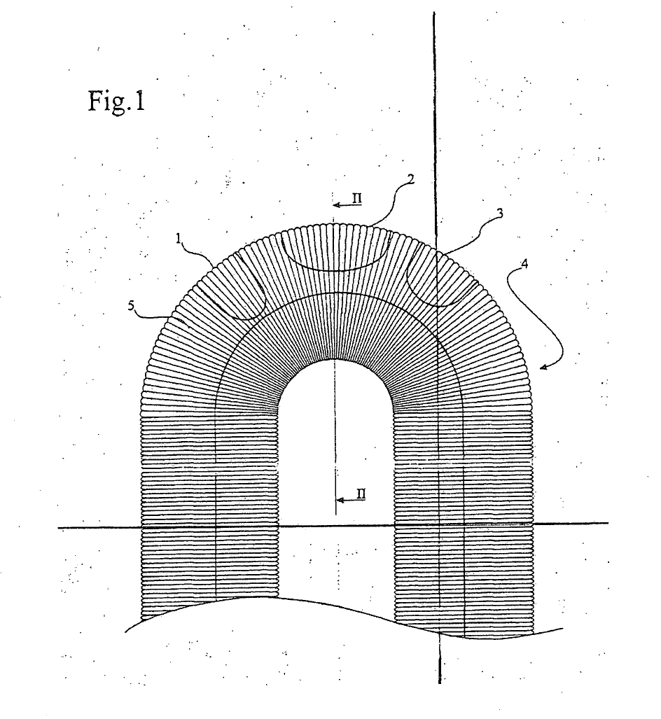

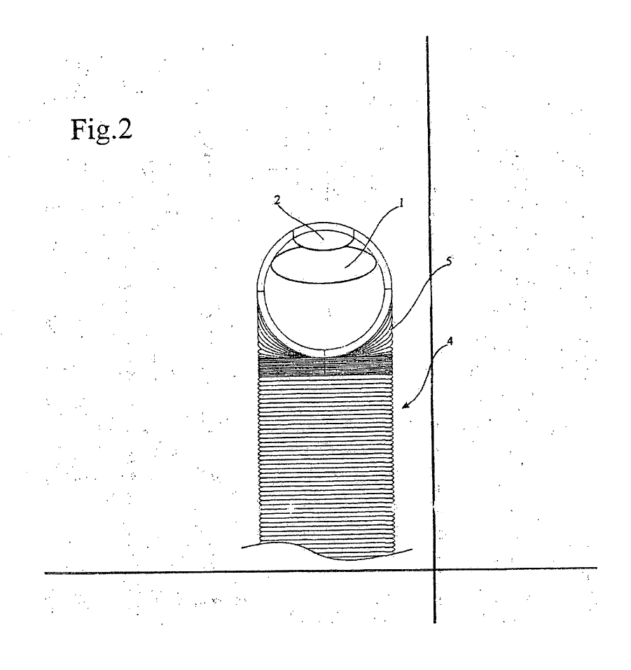

In the structures shown in FIGS. 1 and 2, sinus patches 1, 2, 3 are produced in the form of sections cut from the outer wall in the outer radius of an aortic arch prosthesis 4. The aortic arch prosthesis 4 is a textile aortic arch prosthesis made from a woven or warp-knitted material. It has a pleated construction 5. The aortic arch prosthesis 4 is produced from a tubular, cylindrical prosthesis having pleats. The curved shape has been stabilized by heat-setting the prosthesis in a curved configuration. The aortic arch prosthesis 4 can also be produced from a spray-bonded web, which is formed on an appropriately curved core, which may also be pleated. Sinus patches having, longitudinal pleats (1 and 3) or transverse pleats (2) can be obtained, depending on the method used to cut the sinus patches from the aortic arch prosthesis. Narrow or wide sinus patches can be cut out. Sinus patches having an asymmetrical dome shape can also be produced, depending on the extent to which the side walls of the aortic arch prosthesis are incorporated.

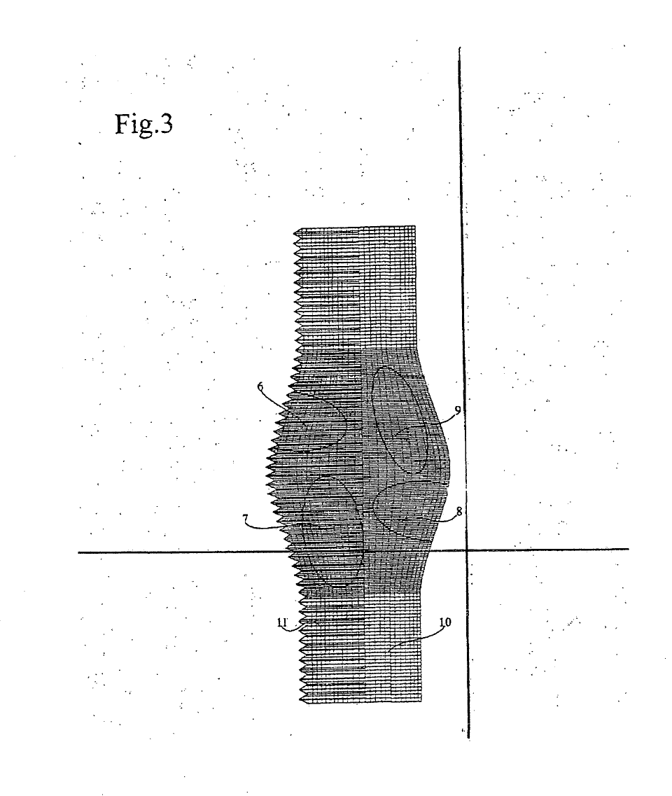

In the structure shown in FIG. 3, sinus patches 6, 7, 8 and 9 are formed as sections cut from a toroidal-shaped vascular prosthesis 10. The vascular prosthesis is preferably a woven vascular prosthesis having a constant number of warp threads over the dome. The vascular prosthesis may have pleats 11, as shown in one of the longitudinal halves. As illustrated, the sinus patches may in turn have transverse pleats or longitudinal pleats, depending on the type of cut-out section. The vascular prosthesis may also be unpleated, as shown in the second longitudinal half. Different shapes and domes are possible here as well, depending on the nature of the cut section. The toroidal-shaped vascular prosthesis can also be produced easily in the form of a spray-bonded web made from polyurethane, for example. The sinus patches are also produced accordingly. It is also possible to produce the dome, especially the toroidal-shaped dome, by stretching and/or shrinking a vascular prosthesis onto a toroidal-shaped core.

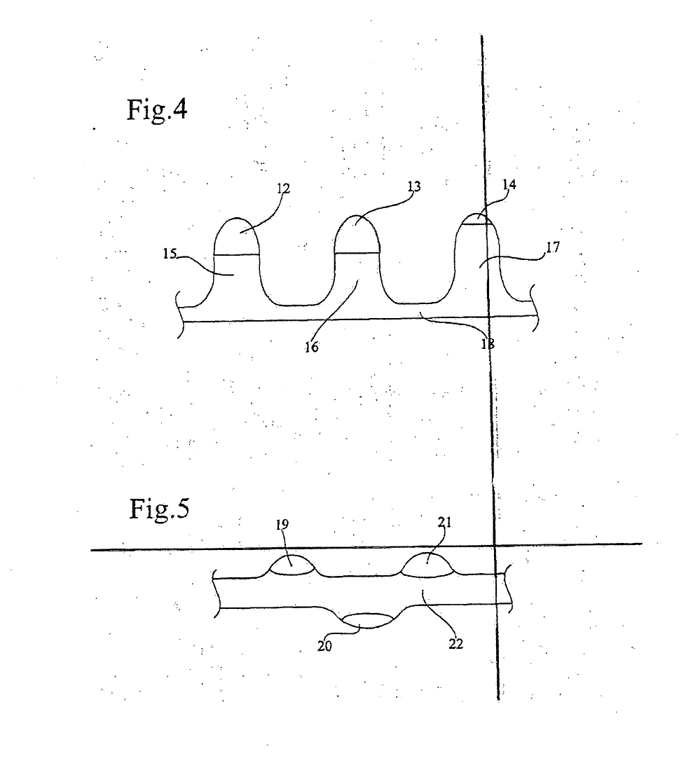

In the structure shown in FIG. 4, sinus patches 12, 13, 14 are provided, which are formed as a polyurethane spray-bonded web. In this case, the dome is obtained by using a base plate 18 having protuberances 15, 16, 17 as the underlay for forming a spray-bonded web. The protuberances 15, 16 and 17 may have different shapes. Accordingly, the sinus patches 12, 13 and 14 also have different shapes. Pleats may also be present here, as shown in the protuberance 16 and the sinus patch 13.

In the structure shown in FIG. 5, sinus patches 19, 20, 21 are provided, which are again formed as a spray-bonded web. In this case, the shape is obtained by spraying a slowly rotating lobed roller 22. Different shapes can also be produced by this method and an arbitrary number of lobes is possible.

The walls of sinus patches 19, 20, 21 can also be formed by dipping the lobed roller into a polyurethane solution and allowing the thus-formed polyurethane layer to dry on the lobed roller while rotating the roller.

With all the structures, the wall material of the sinus patches is already intrinsically dome-shaped from the very beginning, without having to use any special implements to achieve this. This enables any size and shape of sinus patch to be produced or cut out. The sinus patches may be combined with an aortic root prosthesis or may be used to directly replace defective sinuses in the natural aortic root.

1-16. (canceled)

17. A sinus patch for replacing defective sinuses of an aortic root comprising a flat, flexible, domed piece of wall material optionally having pleats and having a three-dimensional dome shape, which is determined by the wall material itself.

18. The sinus patch according to claim 17, formed from a section cut from a three-dimentionally domed vascular prosthesis wall.

19. The sinus patch according to claim 17, formed from a section cut from a wall at an outer side of an aortic arch prosthesis having a fixed curvature that is inherent to the material.

20. The sinus patch according to claim 17, formed by a section cut from a toroidal-shaped vascular prosthesis segment.

21. The sinus patch according to claim 17, made from a textile wall material.

22. The sinus patch according to claim 17, formed from a nonwoven material.

23. The sinus patch according to claim 22, formed from a spray-bonded web.

24. The sinus patch according to claim 17, comprising pleats.

25. The sinus patch according to claim 17, having a length longer than its width.

26. The sinus patch according to claim 24, wherein folds of the pleats run in a longitudinal direction of the sinus patch.

27. The sinus patch according to claim 20, wherein the toroidal-shaped vascular prosthesis segment is made from a woven textile substrate.

28. The sinus patch according to claim 27, wherein the woven textile in the toroidal-shaped vascular segment has a constant number of warp threads.

29. The sinus patch according to claim 27, wherein the toroidal-shaped vascular segment has an increasing and decreasing number of warp threads.

30. The sinus patch according to claim 17, wherein the three-dimensional dome is produced by shrinking and/or stretching a textile wall material.

31. The sinus patch according to claim 17, formed by depositing material onto a three-dimensionally shaped core.

32. The sinus patch according to claim 17, wherein the wall material is porous.
